(19) United States
(12) Patent Application Publication
(10) Pub. No.: US 2021/0063371 A1
(43) Pub. Date: Mar. 4, 2021

(54) GAS-MEASURING SYSTEM AND PROCESS FOR OPERATING A GAS-MEASURING SYSTEM

(71) Applicant: Dräger Safety AG & Co. KGaA, Lubeck (DE)

(72) Inventor: Folke WILL, Lubeck (DE)

(21) Appl. No.: 16/998,732

(22) Filed: Aug. 20, 2020

(30) Foreign Application Priority Data
Aug. 26, 2019 (DE) 10 2019 005 978.6

(51) Int. Cl. G01N 33/00

(52) U.S. Cl. CPC G01N 33/0067; G01N 2033/0068; G01N 33/0027; G01N 33/0063

(57) ABSTRACT

A gas-measuring system (100) measures and outputs a gas concentration and includes a gas sensor (110), a digitization module (120), a network (130), an analysis unit (140) and an output unit (150). The gas sensor outputs a raw data signal (112). The digitization module processes the raw data signal and outputs a corresponding digital sensor signal (124) via a wireless module (126) to the network. The digital sensor signal includes the processed raw data signal and sensor identification information. The analysis unit reads the digital sensor signal from the network, to determine the gas concentration of the gas to be tested based on the measured raw data indicated by the digital sensor signal, and outputs a corresponding digital concentration signal (142) to the network. The output unit reads the digital concentration signal from the network and provides a corresponding output (154) via an output module (152).

### CROSS REFERENCE TO RELATED APPLICATIONS

This application claims the benefit of priority under 35 U.S.C. § 119 of German Application 10 2019 005 978.6, filed Aug. 26, 2019, the entire contents of which are incorporated herein by reference.

### TECHNICAL FIELD

The present invention pertains to a gas-measuring system for measuring and outputting at least a gas concentration of a gas to be tested as well as to a process for operating a gas-measuring system.

### TECHNICAL BACKGROUND

It is known that gas-measuring devices can be connected via a wireless connection to a network with one another. This is especially advantageous in case of the monitoring of areas in which there typically is a risk of exposure to toxic gases and/or to gases that are hazardous to health. Such networks of gas-measuring devices are therefore typically used in locations in which persons may be exposed to hazardous gases over a longer time period, for example, during their work time, for example, in the chemical industry.

Gas-measuring devices typically send measured gas concentrations to a central memory, where these gas concentrations can be analyzed in real time or for a past time interval.

The use of a plurality of gas-measuring devices, the gas concentrations determined by which may provide an overview of monitored areas in which an unusually high level of gas was measured, which makes it possible, for example, to infer a possible leakage, is known as well.

### SUMMARY

An object of the present invention is to make possible an improved gas-measuring system, especially a gas-measuring system with an especially reliable analysis of collected data.

A gas-measuring system for measuring and outputting at least a gas concentration of a gas to be tested, with at least one gas sensor, with at least one digitization module, with a network, with at least one analysis unit and with an output unit is proposed according to the present invention for accomplishing this object.

The at least one gas sensor is configured to output measured raw data via a raw data signal, especially via an analog raw data signal.

The at least one digitization module is arranged in an area surrounding the at least one gas sensor and is configured to receive the raw data signal of the gas sensor, to process, especially to digitize, the raw data signal and to output a corresponding digital sensor signal via a wireless module to the network, wherein the digital sensor signal comprises the processed raw data signal and sensor identification information, and wherein the sensor identification information is configured to make possible an assignment between a processed raw data signal and the corresponding gas sensor.

The at least one analysis unit is configured to read the digital sensor signal from the network, to determine the gas concentration of the gas to be tested based on the measured raw data indicated by the digital sensor signal, and to output a corresponding digital concentration signal to the network. The digital concentration signal comprises here the determined gas concentration and localization information, and the localization information is configured here to make possible an assignment between the determined gas concentration and a location of the corresponding, at least one gas sensor.

The one output unit is configured to read the digital concentration signal from the network and to provide a corresponding output perceptible to a user of the output unit, especially a visual output, via an output module, wherein the perceptible output indicates the determined gas concentration and the localization information.

It was found within the framework of the present invention that the use of different devices for the analysis and for the output of the gas concentration makes possible a separation in space between these two process steps, which makes possible, for example, a central analysis of the measured raw data for a large number of gas sensors and output units. Furthermore, it was found that a decentralized analysis of the raw data within a gas-measuring device is circumvented by the use of raw data, as a result of which especially simple and robust devices, especially gas sensors or a combination of gas sensor and digitization module, can be used within the framework of the gas-measuring system according to the present invention.

The analysis of the raw data can be carried out independently from the time of measurement of the raw data and independently from the time of outputting the determined gas concentrations to the user due to the use according to the present invention of a separate analysis unit. As a result, algorithms and/or devices used for the analysis of the raw data can be changed or repaired, without the ability of the gas sensors or of the output unit to function being limited in the process.

Further, the gas-measuring system according to the present invention makes it possible to sell individual components of the gas-measuring system separately, while the analysis of the measured raw data via the at least one analysis unit continues to be able to be offered as a service for the buyers of the other components and it makes possible as a result a regular improvement of the analysis of the raw data due to advancing technical possibilities, while the other components sold, e.g., gas sensors and analysis units, continue to be able to function without any change.

Furthermore, the multipart structure of the gas-measuring system according to the present invention makes possible a redundant configuration of the system, for example, due to a plurality of gas sensors used, due to a plurality of wireless standards used and/or due to a plurality of analysis units used, as this will be described below within the framework of preferred embodiments.

Furthermore, the gas-measuring system according to the present invention can be embodied in an especially simple manner and at an especially low cost, because existing technological solutions can be used for the individual parts. Thus, the configuration of gas sensors, analysis units and output units is known, in principle, and will not therefore be explained in detail below.

The raw data signal may be an analog or digital raw data signal. The digitization module is preferably configured to carry out an analog-digital conversion of the raw data signal. In exemplary embodiments in which the raw data signal is already a digital raw data signal, the digitization module is correspondingly configured only to provide the digital sensor signal rather than as an analog-digital converter. The processing of the raw data signal correspondingly comprises at least the provision of the digital sensor signal, which indicates the measured raw data from the raw data signal.

The network is a data network according to the present invention.

The perceptible output is preferably an optical output, especially an output via a display. The fact that the perceptible output indicates the determined gas concentration and the localization information means that on the basis of the type or the location of the output, a user of the gas-measuring system is informed of which gas concentration was measured and of the area in which this gas concentration was measured.

The output unit, the at least one gas sensor and the at least one analysis unit are different devices according to the present invention. The at least one gas sensor and the at least one digitization module are arranged in a common housing in an embodiment according to the present invention. In an alternative embodiment according to the present invention, the gas sensor and the digitization module are likewise different devices, i.e., they are arranged in two different housings.

The gas-measuring system according to the present invention makes it advantageously possible to use an especially robust gas sensor. Thus, the gas sensor must only be configured to measure and to output the raw data pertaining to the gas to be measured. This makes possible the use of compact devices, for example, of gas sensors without display and/or without additional control modules. Such compact gas sensors can be manufactured in an especially simple and cost-effective manner.

The at least one digitization module can correspondingly also be manufactured as an especially robust and compact module, because no qualitative analysis of the measured raw data or of the raw data signal has to be carried out.

The gas-measuring system according to the present invention is therefore an especially cost-effective system, in which individual components can function autarchically independently from one another, so that a high level of failure safety can be made possible.

The gas-measuring system according to the present invention may also be a part of a larger system of gas sensors and output units. Gas sensors and output units of different users of the gas-measuring system can thus be combined with one another via a network in one embodiment such that the analysis of the measured raw data is carried out for different users via a single analysis unit or via the same plurality of redundantly configured analysis units.

Preferred embodiments of the gas-measuring system according to the present invention will be described below.

In a preferred embodiment, the at least one digitization module is further configured to receive environmental data, to assign the environmental data to the digital raw data signal and to output them via the digital sensor signal to the network, wherein the environmental data pertain to the at least one gas sensor and wherein the analysis unit is further configured to determine the gas concentration of the gas to be tested on the basis of the measured raw data and of the environmental data received. An especially accurate determination of a gas concentration is possible in this embodiment, because environmental effects can be taken into consideration during the determination of the gas concentration from the measured raw data by the at least one analysis unit. In an especially preferred variant of this embodiment, the environmental data indicate a currently measured temperature, a currently measured ambient pressure, a currently measured air humidity and/or a current position of the at least one gas sensor. The gas concentration can thus be determined on the basis of the temperature, the currently measured ambient pressure, the currently measured air humidity and/or the current position of the gas sensor. The current position of the gas sensor may be used, for example, by the at least analysis unit to infer environmental effects, for example, temperature, ambient pressure and/or air humidity, with a map of the environment received at regular time intervals on the basis of the position of the gas sensor. The current position may be, for example, GPS data.

In a preferred embodiment, the gas-measuring system has at least a first gas sensor and a second gas sensor, wherein the second gas sensor is arranged in the area surrounding the first gas sensor, wherein a respective first digitization module and a second digitization module, which output a corresponding first digital sensor signal and a corresponding second digital sensor signal to the network, are assigned to the gas sensors, and wherein the at least one analysis unit is configured to compare the digital sensor signals received from the network with one another. In a preferred variant of this embodiment, the first gas sensor and the first digitization module are arranged in a common first housing and the second gas sensor and the second digitization module are arranged in a common second housing. The assignment of a gas sensor to exactly one digitization module makes possible an especially simple and robust configuration of the digitization module, because there is no need for receiving and processing a plurality of raw data signals. Furthermore, the provision of two gas sensors in a common environment makes possible an especially high level of failure safety, because another gas sensor can provide measured raw data of the corresponding environment in case of failure of one gas sensor. Furthermore, repair or replacement of a gas sensor is possible in this embodiment without the ability of the entire gas-measuring system to function being compromised.

In an especially preferred variant of the above embodiment, the at least one analysis unit is configured to compare the gas concentrations determined on the basis of the two different digital sensor signals with one another and to provide on the basis of this comparison additional sensor failure information, which indicates whether the digital raw data of the at least two gas sensors, which are located in a common environment, have indicated essentially equal gas concentrations. It can be seen by such a comparison at an early stage when different gas concentrations are determined in an environment. This may be able to be attributed, for example, to a malfunction of one of the two gas sensors. Furthermore, the presence of different gas concentrations may suggest an especially great gradient of the gas concentration. Alarm generation for a user of the gas-measuring system according to the present invention is advantageous in both cases mentioned, so that a checking of the environments of the two gas sensors can be carried out. In one example of this variant, the gas sensor that has determined a gas concentration that deviates the most from a gas concentration determined before is identified as probably being defective based on the sensor failure information.

In another preferred variant of the above embodiment, the gas-measuring system has at least three, preferably at least five, especially at least seven gas sensors in a common environment. There is an especially high level of failure safety in this variant. This may be especially advantageous at locations at which a manual repair or a manual replacement of gas sensors is especially difficult because of special environmental effects, especially based on special meteorological conditions. Furthermore, such a large number of gas sensors in a common environment makes possible an especially accurate determination of a direction from which the gas causing the elevated gas concentration is probably coming.

According to another embodiment, the first gas sensor and the second gas sensor are assigned to a common digitization module, which is configured to output the corresponding first sensor signal and the corresponding second sensor signal to the network. In a variant of this embodiment, the two sensor signals are compared with one another by the analysis unit such that corresponding sensor failure information can be outputted on the basis of this comparison. The first sensor signal and the second sensor signal are preferably outputted via a common wireless module of the common digitization module.

In another preferred embodiment, the analysis unit is configured to determine the localization information based on the sensor identification information. This may be carried out, for example, by an assignment of the sensor identification information to a certain environment or to a concrete location, which assignment is stored in the network. In one embodiment, the localization information can be determined in an especially simple manner and with a small amount of computations.

In another, especially preferred and especially advantageous embodiment, the at least one digitization module of the gas-measuring system according to the present invention has at least a first wireless module and at least a second wireless module and is configured to output the digital sensor signal to the network via the first wireless module corresponding to a first wireless standard and to output the digital sensor signal to the network via the second wireless module corresponding to a second wireless standard, and wherein the first wireless standard is different from the second wireless standard. The wireless connection between the digitization module and the network has an especially fail-safe configuration in this embodiment, because the other wireless module continues to be able to function in case of a defective wireless module. Furthermore, the first wireless standard and the second wireless standard preferably have different specifications, so that the first wireless standard will possibly have the better transmission power at times and the second wireless standard will possibly have the better transmission power at other times, depending on the environment in which the digitization module is located. The transmission of the digital sensor signal to the network is thus made possible in an especially reliable manner. In a preferred variant of this embodiment, the digitization module has at least three different wireless modules with at least three different wireless standards. In another variant of this embodiment, the digital sensor signal is outputted only via the first wireless module with the first wireless standard as long as the wireless module is not defective, especially as long as the digital sensor signal is outputted to the network. The second wireless module and/or the second wireless standard are only used in this variant when the digital sensor signal is not outputted to the network and/or a defect of the wireless module is detected. The wireless standards are, for example, the wireless standards LoRa, LTE, WLAN, ZigBee, BLE and/or Bluetooth, which have already become established on the market.

In another advantageous embodiment, the gas-measuring system according to the present invention has at least two analysis units. The two analysis units are configured in this case to read the digital sensor signal from the network, to determine the gas concentration of the gas to be tested and to output a corresponding digital concentration signal to the network, the respective digital concentration signal indicating the analysis unit that has provided this digital concentration signal. In a variant of this embodiment, one of the at least two analysis units is used during the operation of the gas-measuring system, and it can be determined on the basis of the digital concentration signal which of the two analysis units was used. The second analysis unit is advantageously used in this variant if a defect was determined in the first analysis unit. Both analysis units are used simultaneously with one another in an alternative variant of this embodiment in order to make it possible to compare the digital concentration signals provided by them.

In an especially preferred variant of the above embodiment, the gas-measuring system has, furthermore, a comparison unit, which is configured to receive the at least two digital concentration signals, to compare the respective gas concentrations determined with one another, and to provide an additional analysis failure information, which indicates whether the two gas concentrations determined are essentially identical for the same digital sensor signal. It can advantageously be checked in this variant prior to an output of the gas concentration by the outputting unit whether the at least two analysis units have determined essentially identical gas concentrations and reliable operation of the analysis units can therefore be assumed. A digital concentration signal, which indicates an averaged gas concentration of the two gas concentrations indicated by the two digital concentration signals, is outputted by the comparison unit to the network in an example of this variant.

The at least one analysis unit is configured, furthermore, in another preferred embodiment to output the digital concentration signal such that it comprises, furthermore, alarm information on a configurable threshold value for the outputting of an alarm. In a preferred variant of this embodiment, the output unit is configured to detect the configurable threshold value from the digital concentration signal and to output an alarm depending on the indicated gas concentrations and the detected configurable threshold value, especially to output an alarm if the indicated gas concentration reaches the detected configurable threshold value. In another variant of this embodiment, the configurable threshold value was configured by the analysis unit at an earlier time, for example, it was configured depending on legal standards.

In another preferred embodiment, the output unit is configured to output an alarm signal if the indicated gas concentration reaches a predefined threshold value. The predefined threshold value is preferably stored here in a memory module of the output unit.

The connection between the at least one gas sensor and the at least one digitization module is preferably a wireless connection, for example, a connection by one of the following prior-art wireless standards: LoRa, LTE, WLAN, ZigBee, BLE and/or Bluetooth. As an alternative or in addition, the connection between the at least one gas sensor and the at least one digitization module is a wired connection.

The connection between the at least one analysis unit and the network and/or the connection between the output unit and the network is preferably a wireless connection, for example, a connection by one of the following prior-art wireless standards: LoRa, LTE, WLAN, ZigBee, BLE and/or Bluetooth. As an alternative or in addition, the connection between the at least one analysis unit and the network and/or the connection between the analysis nit and the network is a wired connection.

In another embodiment, the output unit is a mobile device of the user, for example, a smartphone, a tablet or a notebook.

According to another aspect of the present invention, a process is proposed for operating a gas-measuring system to accomplish the above-mentioned object. The process according to the present invention has the following steps:
measurement of raw data and outputting of the measured raw data via a raw data signal;
reception of the raw data signal, processing of the raw data signal into a processed raw data signal and outputting of a corresponding digital sensor signal to a network, wherein the digital sensor signal comprises the processed raw data signal and sensor identification information, wherein the sensor identification information is configured to make possible an assignment between a processed raw data signal and the corresponding gas sensor;
reading of the digital sensor signal from the network, determination of a gas concentration of the gas to be tested based on the measured raw data indicated by the digital sensor signal and outputting of a corresponding digital concentration signal to the network, wherein the digital concentration signal comprises the determined gas concentration and localization information, and wherein the localization information is configured to make possible an assignment between determined gas concentration and a location of the corresponding, at least one gas sensor; and
reading of the digital concentration signal from the network and provision of a corresponding perceptible output, especially visual output, wherein the perceptible output indicates the determined gas concentration and the localization information.

The process according to the present invention advantageously makes possible the central determination of the gas concentration and the use of a plurality of individual devices connected to the network. Due to the fact that a network can be provided in a reliable manner, the individual processing steps, namely, the measurement of the raw data, the determination of the gas concentration and the provision of the output can take place independently from one another in terms of time.

The process according to the present invention can thus be used in an especially robust manner, because, for example, gas concentrations already determined continue to be provided by the network, for example, also in case of a temporary absence of measurement of raw data, e.g., during a repair or a replacement of a gas sensor. In addition, a malfunction occurring during the output of gas concentrations or during the determination of the gas concentration does not lead to an impairment during the measurement of the raw data, because the measured raw data continue to be stored as digital sensor signal in the network.

In an especially advantageous embodiment of the process according to the present invention, a respective process step is carried out several times by different devices, and the results from these devices are compared with one another in a subsequent process step, so that a malfunction of a device can be inferred.

In an especially preferred embodiment, provisions are made for the multiple measurement of the raw data, for the multiple outputting of the digital sensor signal to the network and/or for the multiple determination of the gas concentration. An especially high level of failure safety can be made possible by the process according to the present invention due to such a redundancy.

The present invention shall be explained in more detail on the basis of advantageous exemplary embodiments shown schematically in the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of a first exemplary embodiment of a gas-measuring system according to the present invention;

FIG. 2 is a schematic view of a second exemplary embodiment of the gas-measuring system according to the present invention;

FIG. 3 is a schematic view of a third exemplary embodiment of the gas-measuring system according to the present invention;

FIG. 4 is a schematic view of a fourth exemplary embodiment of the gas-measuring system according to the present invention;

FIG. 5 is a schematic view of a fifth exemplary embodiment of the gas-measuring system according to the present invention; and

FIG. 6 is a flow chart of an exemplary embodiment of a process according to another aspect of the present invention.

### DESCRIPTION OF PREFERRED EMBODIMENTS

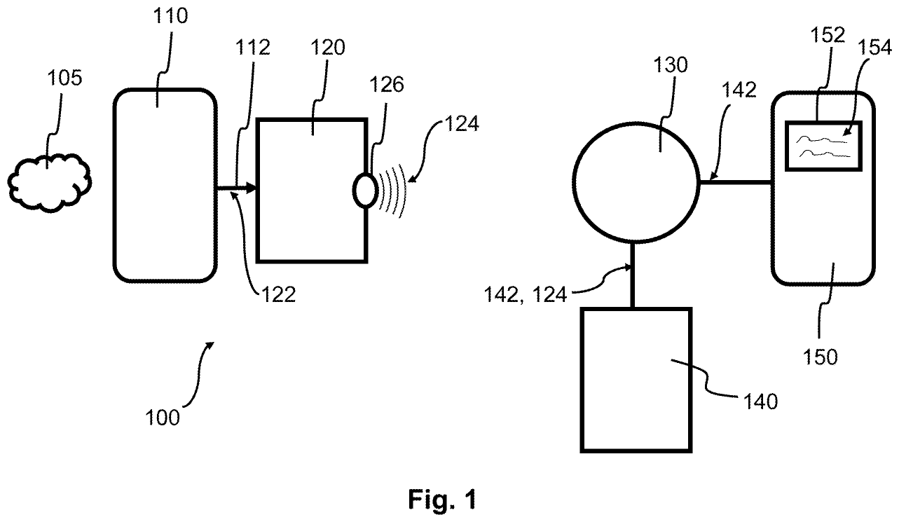

Referring to the drawings, FIG. 1 shows a schematic view of a first exemplary embodiment of a gas-measuring system 100 according to the present invention.

The gas-measuring system 100 for measuring and outputting at least a gas concentration of a gas 105 to be tested comprises at least one gas sensor 110, at least one digitization module 120, a network 130, at least one analysis unit 140 and an output unit 150.

The at least one gas sensor 110 is configured to measure gas concentration and to output measured raw data via a raw data signal 112. The measured raw data pertain to the gas 105 to be tested. There is exactly one gas sensor 110 in the exemplary embodiment being shown. The gas sensor 110 preferably has an especially simple configuration and has no operating elements and no visual output elements. In the exemplary embodiment shown, the gas sensor 110 has only an LED (not shown), which signals by blinking that the gas sensor 110 is in operation. The general configuration of a gas sensor is known and will not therefore be explained in detail below.

The at least one digitization module 120 is arranged in an area surrounding the at least one gas sensor 110. The digitization module 120 is in this case a separate device, which is arranged directly at a gas sensor and which is connected by a line 122 to the gas sensor 110. In another exemplary embodiment, the digitization module and the gas sensor are arranged in a common housing. The at least one digitization module 120 is configured to receive the raw data signal 112 of the gas sensor 110 via the line 122, to process the raw data signal 112 into a processed raw data signal and to output a corresponding digital sensor signal 124 to the network 130 via a wireless module 126. The wireless module 126 uses the wireless standard WLAN in the exemplary embodiment shown. In other additional or alternative exemplary embodiments, the wireless module uses the wireless standard LoRa, LTE, ZigBee, BLE and/or Bluetooth. The digital sensor signal 124 comprises the processed raw data signal and sensor identification information, the sensor identification information being configured to make possible an assignment between a processed raw data signal and a corresponding gas sensor 110. The sensor identification information is preferably stored in a memory of the digitization module 120. The gas sensor 110, whose raw data are contained in the digital sensor signal 124, can thus be inferred on the basis of the digital sensor signal 124 being stored in the network 130. An assignment between sensor identification information and the location of the sensor is preferably stored in a memory of the gas-measuring system 100, for example, within a memory unit connected to the network 130.

The at least one analysis unit 140 is configured to read the digital sensor signal 124 from the network 130, to determine the gas concentration of the gas to be tested on the basis of the measured raw data indicated by the digital sensor signal 124 and to output a corresponding digital concentration signal 142 to the network 130. The digital concentration signal 142 comprises in this case the determined gas concentration and localization information, the localization information being configured to make possible an assignment between the determined gas concentration and a location of the corresponding, at least one gas sensor 110. The determination of the gas concentration from the measured raw data is carried out according to a predefined algorithm, which is stored on the analysis unit 140. The reading of the digital sensor signal 124 and the outputting of the digital concentration signal 142 are preferably carried out in an automated manner. Manual operation of the analysis unit 140 is preferably provided according to the present invention only for changing the algorithm for analyzing the measured raw data or for resetting other boundary conditions, for example, alarm limits. The analysis unit 140 may be, for example, a computer comprising one or more processors, which can access the network 130 via a WLAN connection. In other exemplary embodiments, the connection to the network 130 is established in a wired manner or via a wireless standard, the wireless standard being LoRa, LTE, BLE, ZigBee and/or Bluetooth. The localization information is determined in this case especially advantageously on the basis of the sensor identification information. This is carried out by a predefined assignment of a location being monitored by the gas sensor to sensor identification information. The sensor identification information is preferably a sequence of symbols comprising letters, numbers and/or special symbols.

The output unit 150 is configured to read the digital concentration signal 142 from the network 130 and to provide a corresponding output 154 perceptible for a user of the output unit 150, especially a visual output 154. The perceptible output 154 indicates the determined gas concentration and the localization information. The output module 152 comprises in this case a display, via which the visual output 154 is provided. The output 154 displays in this case the gas concentration numerically and displays by means of a number known to the user a location at which this gas concentration was measured. In one exemplary embodiment, not shown, the gas concentration is outputted via a color chart, which shows based on the displayed color whether the current gas concentration is a critical gas concentration and/or whether a change has taken place in the gas concentration within a predefined past time period.

The gas-measuring system 100 according to the present invention makes possible a repair and/or a replacement of the gas sensor 110 without the ability of the other components of the gas-measuring system 100 to function being compromised. Furthermore, the central analysis via the analysis unit 140 makes it possible to change the underlying analysis algorithm for the measured raw data, without this requiring any action to be performed on the gas sensor 110, the digitization module 120 or the output unit 150.

The analysis unit 140 can be advantageously regularly updated and especially adapted to current legal specifications by a service provided, while, for example, the gas sensor 110 and the output unit 150 do not require any updating or do require updating much more rarely compared to the analysis unit 140.

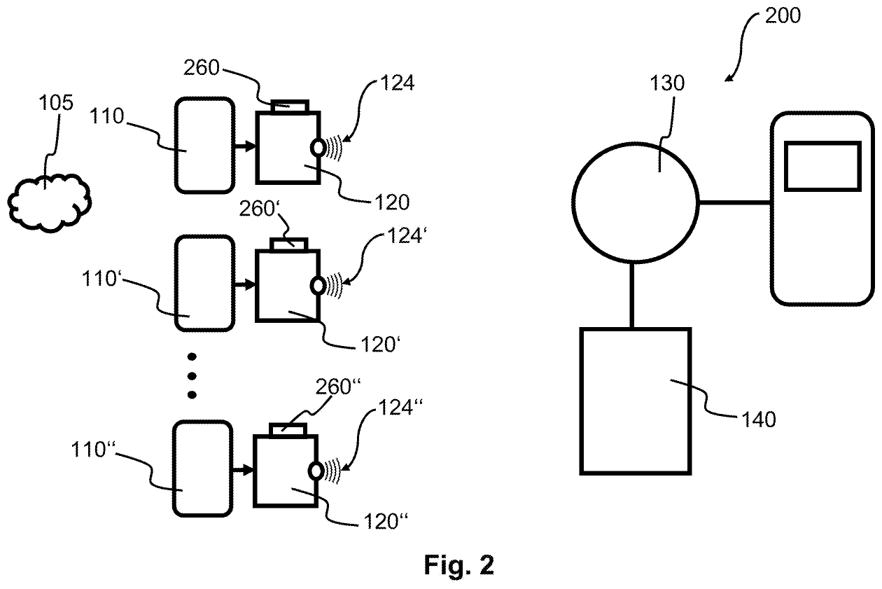

FIG. 2 shows a schematic view of a second exemplary embodiment of the gas-measuring system 200 according to the present invention.

The gas-measuring system 200 differs from the gas-measuring system 100 shown in FIG. 1 in that it has a plurality of gas sensors 110, 110′, 110″ with a correspondingly assigned plurality of digitization modules 120, 120′, 120″.

The plurality of digitization modules 120, 120′, 120″ output a plurality of digital sensor signals 124, 124, 124″ to the network 130 corresponding to the assigned plurality of gas sensors 110, 110′, 110″. The plurality of gas sensors 110, 110′, 110′ are arranged at least partly in the same environment.

The analysis unit 140 is additionally configured in the exemplary embodiment being shown to compare to one another the determined gas concentrations that are assigned to the raw data that were measured in the same environment. This is carried out on the basis of the determined localization information and/or on the basis of the read sensor identification information. A defect of a gas sensor 110, 110′, 110″ is detected based on this comparison. Failure information, which indicates whether the digital raw data of the gas sensors located in a common environment have indicated essentially the same gas concentrations, is thus provided in this exemplary embodiment. This failure information is typically noticeable for a user of the gas-measuring system, for example, by an alarm provided via the output unit, only if the measured gas concentrations differ from one another within a common environment. In one exemplary embodiment, not shown, the gas concentrations of the raw data measured in the same environment are averaged in order to obtain an average value for the gas concentration in this environment.

In one exemplary embodiment, not shown, at least two gas sensors are assigned to a digitization module, so that a digitization module outputs at least two different digital sensor signals to the network via the wireless module.

Finally, the gas-measuring system 200 also differs from the gas-measuring system 100 in that the plurality of digitization modules 120, 120′, 120″ further comprise a respective environmental data module 260, 260′, 260″. The environmental data module 260, 260′, 260″ is configured to provide environmental data. The environmental data are stored here on the environmental data module 260, 260′, 260″, they are received by this module and/or they are measured by this module. A respective environmental data module 260, 260′, 260″ may comprise a plurality of individual modules, which are configured for the provision of concrete environmental data. Furthermore, each digitization module from the plurality of digitization modules 120, 120′, 120″ is configured to receive the environmental data from the respective environmental module 260, 260′, 260″, to assign these environmental data to the digital raw data signal and to output them via the respective digital sensor signal 124, 124′, 124″ to the network 130. The environmental data pertain here to the respective environment of a gas sensor 110, 110′, 110″. The analysis unit 140 is further configured here to determine the gas concentration of the gas 105 to be tested on the basis of the measured raw data and of the received environmental data.

The environmental data indicate here a currently measured temperature, a currently measured ambient pressure, a currently measured air humidity and/or a current position of the gas sensors 110, 110′, 110″.

The concrete configuration of suitable measuring devices for embodying such an environmental data module 260, 260′, 260″ is basically known and will not therefore be explained in detail below.

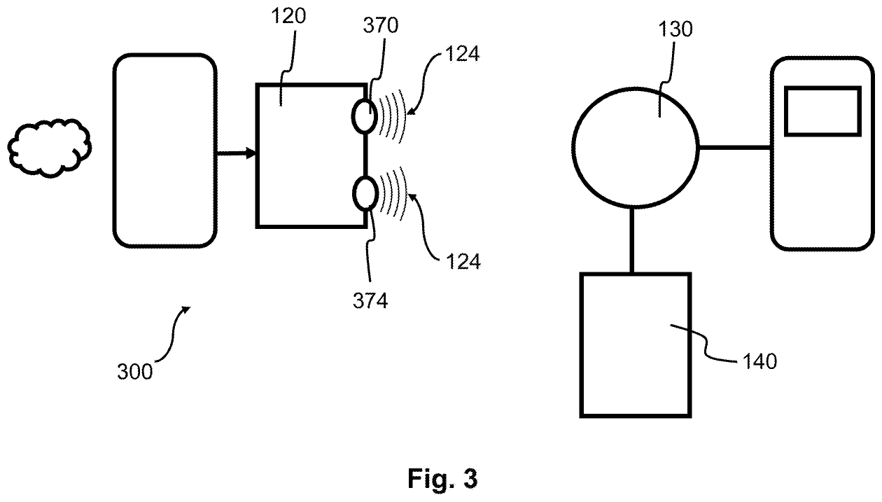

FIG. 3 shows a schematic view of a third exemplary embodiment of the gas-measuring system 300 according to the present invention.

The gas-measuring system 300 differs from the gas-measuring system 100 shown in FIG. 1 only in that the digitization module 120 has at least a first wireless module 370 and a second wireless module 374. The digital sensor signal 124 is outputted to the network 130 via the first wireless module 370 corresponding to a first wireless standard and the digital sensor signal is outputted to the network 130 via the second wireless module 374 corresponding to a second wireless standard, the two wireless standards being different from one another. The two wireless standards are a WLAN connection and a Bluetooth connection in the exemplary embodiment being shown.

However, any combination of common wireless standards, e.g., a combination of at least two of the wireless standards LoRa, LTE, WLAN, ZigBee, BLE or Bluetooth, is basically conceivable.

In one exemplary embodiment, not shown, the digitization module is configured to output signals corresponding to three different wireless standards via three different wireless modules.

The analysis unit 140 is configured in the exemplary embodiment shown to process further the digital sensor signal 124 that is present in the network 130 with the best quality, for example, with the best signal-to-noise ratio or with the largest volume of data.

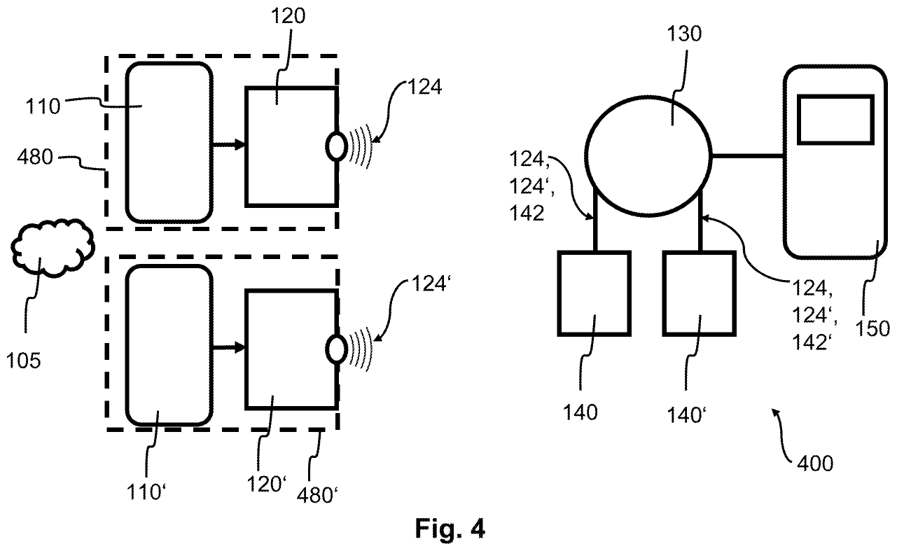

FIG. 4 shows a schematic view of a fourth exemplary embodiment of the gas-measuring system 400 according to the present invention.

The gas-measuring system 400 differs from the gas-measuring system 200 shown in FIG. 2 in that exactly two different gas sensors 110, 110′ with corresponding two assigned digitization modules 120, 120′ are provided, a respective gas sensor 110, 110′ and a respective digitization module 120, 120′ having a common housing 480, 480′. As a result, these two components of the gas-measuring system 400 according to the present invention can be arranged in an area with a gas concentration to be monitored in an especially compact and simple manner.

Furthermore, the gas-measuring system 400 differs from the gas-measuring system 200 in that two different analysis units 140, 140′ are provided in the gas-measuring system 400. The two analysis units 140, 140′ are configured each to read the two digital sensor signals 124, 124′ from the network 130, to determine the respective gas concentration of the gas 105 to be tested and to output each a corresponding digital concentration signal 142, 142′ to the network. The digital concentration signal 142, 142′ indicates here the analysis unit 140, 140′ that has provided this digital concentration signal 142, 142′. The analysis of the two digital sensor signals 124, 124′ from the same environment is carried out here as was described in connection with FIG. 2.

A malfunction of an analysis unit 140, 140′ can be detected in the exemplary embodiment at an early stage by the fact that different gas concentrations were determined. The output unit 150 is preferably configured in this exemplary embodiment to detect a difference between the determined gas concentrations for a common set of measured raw data and to output a corresponding warning signal to a user of the output unit 150 and/or to the network 130. In case of an output to the network 130, the two analysis units 140, 140′ are preferably configured, furthermore, to receive the corresponding warning signal and to output it to an operator of the analysis units 140, 140′.

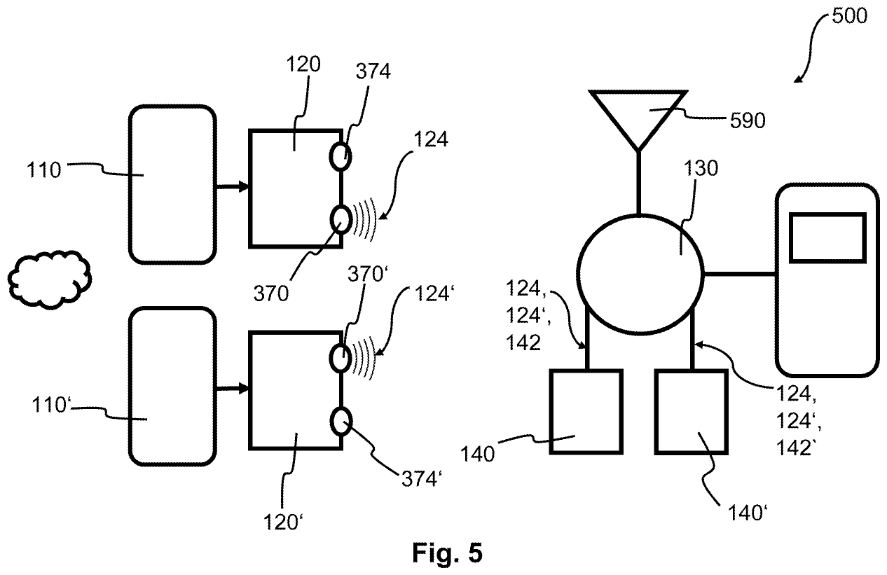

FIG. 5 shows a schematic view of a fifth exemplary embodiment of the gas-measuring system 500 according to the present invention.

The gas-measuring system 500 differs from the gas-measuring system 400 shown in FIG. 4 in that the two digitization modules 120, 120′ have, separated from the two gas sensors 110, 110′, separate housings, and, as was already described in connection with FIG. 3, a first wireless module 370, 370′ and a second wireless module 374, 374′ each.

Unlike as explained in connection with FIG. 3, the two digitization modules 120,120′ are configured to output the respective digital sensor signal 124, 124′ exclusively via the respective first wireless module 370, 370′ corresponding to the first wireless standard. It is only when a disturbance was detected during the outputting of the respective digital sensor signal 124, 124′, for example, by the analysis unit 140, 140′, that the output is changed over by the respective digitization module 120, 120′ such that the digital sensor signal 124, 124′ is outputted to the network 130 via the respective second wireless module 374, 374′ corresponding to the second wireless standard. In a preferred variant of this exemplary embodiment, each digitization module 120, 120 is changed over to another wireless standard, if such other wireless standard is available, if a problem is detected with one wireless standard.

Furthermore, the gas-measuring system 500 differs from the gas-measuring system 400 in that the gas-measuring system 500 additionally has a comparison unit 590. The comparison unit 590 is configured to read at least two digital concentration signals 142, 142′ from the network, to compare the respective determined gas concentrations with one another, and to provide additional analysis failure information. The analysis failure information shows whether the two determined gas concentrations are essentially identical for the same digital sensor signal 124, 124′. The output unit 150 continues to be configured in the exemplary embodiment shown to read the analysis failure information and to inform a user about a discrepancy in the determination of the gas concentration by the two analysis units 140, 140′. In one exemplary embodiment, not shown, the two analysis units 140, 140′ are configured to read the analysis failure information and to output a corresponding warning. In one exemplary embodiment, not shown, the gas-measuring system has at least three analysis units, and especially at least five analysis units. More than two analysis units simplify the determination of the analysis unit that has determined an incorrect gas concentration, because a plurality of other analysis units have determined a different result.

The possibilities of configuring the gas-measuring system according to the present invention in a redundant manner, which were shown in the exemplary embodiments, may obviously be combined. The provision of a plurality of analysis units, of a plurality of gas sensors, of a plurality of wireless modules with corresponding wireless standards and/or of a plurality of wireless modules leads to an increase in failure safety and it leads, both individually and in combination with one another, to a gas-measuring system according to the present invention.

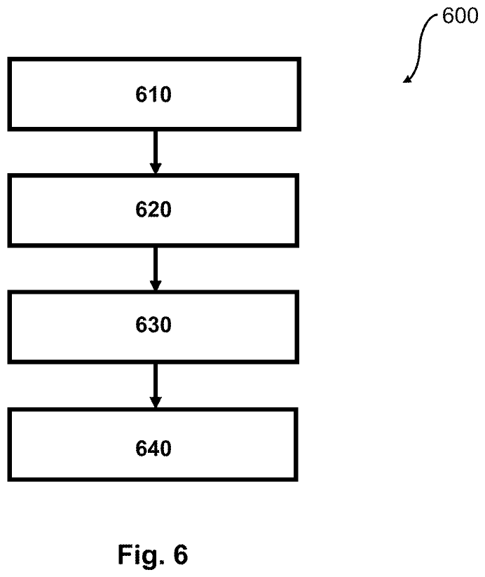

FIG. 6 shows a flow chart of an exemplary embodiment of a process 600 according to another aspect of the present invention.

The process 600 according to the present invention for operating a gas-measuring system has the process steps described below.

A first step 610 comprises a measurement of raw data and the outputting of the measured raw data via a raw data signal.

A next step 620 comprises the reception of the raw data signal, the processing of the raw data signal into a processed raw data signal and the outputting of a corresponding digital sensor signal to a network, the digital sensor signal comprising the processed raw data signal and sensor identification information, the sensor identification information being configured to make possible an assignment between the processed raw data signal and the corresponding gas sensor.

A next step 630 comprises reading of the digital sensor signal from the network, determination of a gas concentration of the gas to be tested based on the measured raw data indicated by the digital sensor signal and outputting of a corresponding digital concentration signal to the network, the digital concentration signal comprising the determined gas concentration and localization information, and the localization information being configured to make possible an assignment between the determined gas concentration and a location of the corresponding, at least one gas sensor.

A final step 640 comprises the reading of the digital concentration signal from the network and the provision of a corresponding perceptible output, the perceptible output indicating the determined gas concentration and the localization information.

Steps 610 and 620 are preferably carried out immediately following one another in this order. Step 610 is preferably carried out automatically at regular intervals. Time intervals of at least 2 sec, preferably at least 10 sec and especially preferably at least 1 min are provided for the measurement of gases in a possibly gas-exposed environment.

Steps 620, 630 and 640 are carried out according to the present invention in this order, and there may be long time intervals between these steps. Thus, an output of the determined gas concentrations according to step 640 may not take place even over several days, so that it is only after some time that the gas concentrations are checked by a user of the analysis unit for the past time. In particular, the measurement and the outputting of the raw data according to step 610 and step 620 are independent from the outputting of the determined gas concentrations according to step 640. The only precondition for the output according to step 640 is that an analysis of the measured raw data shall have taken place in the meantime after step 630. One of these process steps may also be delayed by the circumstance that the corresponding device is replaced, repaired or subjected to another maintenance.

The measurement and the outputting of the raw data according to step 610 and step 620 are preferably configured redundantly, so that an incorrect measurement and also an incorrect outputting can be detected in a short time. Furthermore, the analysis of the measured raw data according to step 630 is preferably configured redundantly, so that an incorrect analysis of the measured raw data can be detected in a short time.

While specific embodiments of the invention have been shown and described in detail to illustrate the application of the principles of the invention, it will be understood that the invention may be embodied otherwise without departing from such principles.

### LIST OF REFERENCE NUMBERS

100, 200, 300, 400, 500 Gas-measuring system

105 Gas

110, 110′, 110″ Gas sensor

112 Raw data signal

120, 120′, 120″ Digitization module

122 Line

124, 124′, 124″ Digital sensor signal

126 Wireless module

130 Network

140, 140′ Analysis unit

142, 142′ Digital concentration signal

150 Output unit

152 Output module

154 Output

260, 260′, 260″ Environmental data module

370 First wireless module

374 Second wireless module

480 Housing

590 Comparison unit

600 Process

610, 620, 630, 640 Process steps

What is claimed is:

1. A gas-measuring system for measuring and outputting at least a gas concentration of a gas to be tested, the gas-measuring system comprising:
a gas sensor configured to output measured raw data via a raw data signal;
a digitization module arranged in an area adjacent to the gas sensor and configured to receive the raw data signal of the gas sensor, to process the raw data signal and to output a corresponding digital sensor signal via a wireless module to a network, wherein the digital sensor signal comprises the processed raw data signal and sensor identification information, and the sensor identification information is configured to make possible an assignment between the processed raw data signal and the corresponding gas sensor;
an analysis unit configured to read the digital sensor signal from the network, to determine the gas concentration of the gas to be tested based on the measured raw data indicated by the digital sensor signal and to output a corresponding digital concentration signal to the network, wherein the digital concentration signal comprises the determined gas concentration and localization information, and the localization information is configured to make possible an assignment between a determined gas concentration and a location of the corresponding gas sensor; and
an output unit configured to read the digital concentration signal from the network and to provide a corresponding output perceptible for a user of the output unit, wherein the perceptible output indicates the determined gas concentration and the localization information.

2. A gas-measuring system in accordance with claim 1, wherein:
the digitization module is further configured to receive environmental data, to assign the environmental data to the digital raw data signal and to output same via the digital sensor signal to the network;
the environmental data pertain to an environment of the at least one gas sensor; and
the analysis unit is further configured to determine the gas concentration of the gas to be tested on the basis of the measured raw data and of the received environmental data.

3. A gas-measuring system in accordance with claim 2, wherein the environmental data indicate at least one of a currently measured temperature, a currently measured ambient pressure, a currently measured air humidity and a current position of the at least one gas sensor.

4. A gas-measuring system in accordance with claim 1, wherein:
the gas sensor is a first gas sensor and further comprising a second gas sensor arranged in an area surrounding the first gas sensor;
the digitization module is a first digitization module and further comprising a second digitization module, the first digitization module outputting the digital sensor signal as a corresponding first digital sensor signal and the second digitization modules outputting a corresponding second digital sensor signal to the network;
the first digitization module and the second digitization module are assigned to the first and second gas sensors; and
the at least one analysis unit is configured to compare the digital sensor signals received from the network with one another.

5. A gas-measuring system in accordance with claim 4, wherein the analysis unit is configured to compare the gas concentrations determined on the basis of the two different digital sensor signals with one another and to provide, based on this comparison, additional sensor failure information, which indicates whether the digital raw data of the at least two gas sensors, which are located in a common environment, have indicated essentially identical gas concentrations.

6. A gas-measuring system in accordance with claim 1, wherein the analysis unit is configured to determine the localization information on the basis of the sensor identification information.

7. A gas-measuring system in accordance with claim 1, wherein:
the digitization module comprises a first wireless module and a second wireless module and is configured to output the digital sensor signal via the first wireless module corresponding to a first wireless standard to the network and to output the digital sensor signal via the second wireless module corresponding to a second wireless standard to the network; and
the first wireless standard is different from the second wireless standard.

8. A gas-measuring system in accordance with claim 4, further comprising another analysis unit, to provide at least two analysis units that are configured to read each digital sensor signal from the network, to each determine the gas concentration of the gas to be tested and each output a corresponding digital concentration signal to the network, wherein the respective digital concentration signal indicates the analysis unit that has provided the digital concentration signal.

9. A gas-measuring system in accordance with claim 8, further comprising a comparison unit configured to receive the at least two digital concentration signals, to compare the respective determined gas concentrations with one another and to provide additional analysis failure information, which indicates whether the two determined gas concentrations for the same digital sensor signal are essentially identical.

10. A process for operating a gas-measuring system, the process comprising the steps of:
measuring with a sensor and providing measured raw data and outputting the measured raw data via a raw data signal;
receiving the raw data signal and processing the raw data signal into a processed raw data signal and outputting a corresponding digital sensor signal to a network, wherein the digital sensor signal comprises the processed raw data signal and sensor identification information, wherein the sensor identification information is configured to make possible an assignment between a processed raw data signal and a corresponding gas sensor;
reading of the digital sensor signal from the network and determining a gas concentration of a gas to be tested based on the measured raw data indicated by the digital sensor signal and outputting a corresponding digital concentration signal to the network, wherein the digital concentration signal comprises the determined gas concentration and localization information, and wherein the localization information is configured to make possible an assignment between a determined gas concentration and a location of the corresponding, at least one gas sensor; and
reading the digital concentration signal from the network and providing a corresponding perceptible output, wherein the perceptible output indicates the determined gas concentration and the localization information.
